(19) United States
(12) Patent Application Publication
(10) Pub. No.: US 2012/0271330 A1
(43) Pub. Date: Oct. 25, 2012

(54) TEMPLATE FOR BIOLOGIC OR SYNTHETIC GRAFT PREPARATION

(76) Inventor: Corey Robert WILSON-WIRTH, San Diego, CA (US)

(21) Appl. No.: 13/272,921

(22) Filed: Oct. 13, 2011

Related U.S. Application Data
(60) Provisional application No. 61/392,850, filed on Oct. 13, 2010.

(30) Foreign Application Priority Data
Feb. 11, 2011 (FR) 1100431

(51) Int. Cl. A61B 17/04

(52) U.S. Cl. 606/148

(57) ABSTRACT

A device for peroperative preparation of an implantable patch with suturing according to embodiments of the present invention includes a first element having one or more first through-openings configured to receive a suture, a second element movable with respect to the first element, the second element including one or more second through-openings configured to receive the suture, and a mechanism configured to position the first and second elements with respect to each other in a closed configuration in which at least one of the one or more first through-openings is aligned with at least one of the one or more second through-openings and in which the first and second elements are close enough together to hold the implantable patch between the first and second elements.

### CROSS-REFERENCE TO RELATED APPLICATION

This application claims the benefit of U.S. Provisional Patent Application Ser. No. 61/392,850, filed on Oct. 13, 2010, and claims foreign priority to French Patent Application No. FR 1100431, filed on Feb. 11, 2011, both of which are incorporated by reference herein in their entireties for all purposes.

### TECHNICAL FIELD

Embodiments of the present invention relate to a mechanism and a system for peroperative preparation of an implantable patch with suture.

### BACKGROUND

In practice, in order to be effective, implantable patches must be fixed in a stable and permanent manner to the anatomical tissues of a patient. To do this, surgeons use sutures which they pass through the patch and which they connect to the patient's tissues. If necessary, the sutures cooperate with anchors implanted in the bone and/or are knotted with various types of stitching. As long as the patch is sufficiently small in size such that it only requires one or two sutures, the surgeons can prepare the patch in situ, in immediate proximity with the tissues of the patient in whom the patch is going to be sutured, after having introduced the patch into the patient's body.

However, when the patch is a little bit larger and/or must be fixed with a greater number of sutures, the surgeons have no choice but to prepare the patch on the outside of the patient's body, typically in a sterile field defined by an intervention table dedicated to a surgical unit. In fact, the preparation of patches for implantation is performed during the operation by the surgeon, and the surgeon must often access the implantation site of the patient in order to best estimate the placement of the sutures desired for fixation of the patch. Furthermore, during the preparation of the patch, just before implantation, the surgeon must often resize the patch, in the sense that he or she must cut a smaller piece from a piece of relatively larger dimensions, which becomes the patch for implantation, of which the dimensions and form are adapted to the needs of the current surgical procedure. As a result, during the course of such procedure, the surgeon encounters challenges in manipulating the patch for resizing it by cutting, either with the help of a pair of scissors which may leave the patch edges with an irregular cut edge, or with the help of a scalpel which requires that the patch be well-secured before cutting. Then the surgeon must often pass across the patch several strands of suture, which often requires that the patch be held and firmly stretched, typically with the help of a hemostat, during which the surgeon pushes across the patch suturing needles for the suture strands.

In addition, each of these fastidious manipulations described above must be performed in as sterile a manner as possible, as the patch is typically provided in a sterile condition at the beginning of the surgery.

### SUMMARY

Some embodiments of the present invention maintain a patch in place between two opposed elements, by “sandwiching” the patch between the two elements, without damaging the patch with an excess immobilization force between the two elements. To do this, the device according to embodiments of the present invention includes a mechanism which permits the closing of the two elements and securing them in place with respect to each other with the patch between. In certain cases, the two elements may be connected to remain positioned with respect to each other, while also being easily manipulated together. The two elements of the device may be perforated at least partially in a manner permitting the successive introduction of suture through one of the two elements, through the patch captured between the two elements, and through the second element. According to some embodiments of the present invention, each of the two elements of the device includes a plurality of through-holes or through-openings configured to receive a suture, which permit the surgeon to choose the placement of suture passage, without having to modify the relative positioning between the patch and the two elements. The device according to embodiments of the present invention also permits the placement of sutures through the patch in a rapid and precise manner, in particular using suture pullers or suturing needles, since such needles are guided through the through-holes of two elements of the device while passing through the patch.

According to some embodiments of the present invention, the device also permits the placement of sutures which are completely independent; in other words, (a) the suture which, having been passed through the patch, is not connected to anything or (b) the suture which has already been connected to an object other than the patch, such as a suture which has already been passed through or tied to a patient's anatomic tissues before passage through the patch.

A device for peroperative preparation of an implantable patch with suturing, according to embodiments of the present invention, includes a first element with one or more first through-openings configured to receive a suture, a second element movable with respect to the first element, the second element including one or more second through-openings configured to receive the suture, and a mechanism configured to position the first and second elements with respect to each other in a closed configuration in which at least one of the one or more first through-openings is aligned with at least one of the one or more second through-openings and in which the first and second elements are close enough together to hold the implantable patch between the first and second elements. According to some embodiments of the present invention, one or both of the first and second elements is formed of a transparent or translucent material. At least one of the one or more first through-openings and the at least one of the one or more second through-openings may have a closed shape. The at least one of the one or more first through-openings and the at least one of the one or more second through-openings may have an open shape and open toward an outer perimeter of the first or second elements, according to embodiments of the present invention.

According to some embodiments of the present invention, the first element includes a first border portion, the second element includes a second border portion configured for alignment with the first border portion in the closed configuration, wherein the at least one of the one or more first through-openings has an open shape and opens toward the first border portion, and wherein the at least one of the one or more second through-openings has an open shape and opens toward the second border portion.

According to some embodiments of the present invention, the at least one of the one or more first through-openings and the at least one of the one or more second through-openings are elongated slots, each of which may have a length and be graduated along the length at spaced intervals. The one or more first through-openings may be a plurality of first through-openings arranged on the first element according to a predetermined pattern, and the first element may include markings relating at least a portion of the plurality of first through-openings to the predetermine pattern to assist a surgeon in sizing the implantable patch, according to embodiments of the present invention. In some cases, the first element includes a first face, the second element includes a second face, and the first and second faces are substantially planar and in contact with the implantable patch in the closed configuration, according to embodiments of the present invention.

According to some embodiments of the present invention, one of the first and second faces comprises an abutment edge configured to position the implantable patch with respect to the one or more first through-openings and the one or more second through-openings when an edge of the implantable patch is aligned with the abutment edge in the closed configuration. At least one of the first and second faces may include retention features configured to retain the implantable patch with friction in the closed configuration. In some cases, both the first and second faces include the retention features, and the retention features are projecting barbs distributed across at least a majority of the first and second faces, according to embodiments of the present invention. The first face may have a first outer perimeter, the second face may have a second outer perimeter, and the second face may not be completely covered by the first face in the closed configuration, such that the first outer perimeter is configured to guide a cutting tool along the implantable patch in the closed configuration.

Some embodiments of the present invention may include a locking mechanism, for example a hinge, configured to lock the first and second elements in the closed configuration.

A system for peroperative preparation of an implantable patch with suturing according to embodiments of the present invention includes a first element with one or more first through-openings configured to receive a suture, a second element movable with respect to the first element, the second element with one or more second through-openings configured to receive the suture, a mechanism configured to position the first and second elements with respect to each other in a closed configuration in which at least one of the one or more first through-openings is aligned with at least one of the one or more second through-openings and in which the first and second elements are close enough together to hold the implantable patch between the first and second elements, the suture, and at least one needle configured for placement through the at least one of the one or more first through-openings, through the implantable patch, and through the at least one of the one or more second through-openings.

A device for peroperative preparation of an implantable patch with suturing according to embodiments of the present invention includes a first element with one or more first through-openings configured to receive a suture, a second element movable with respect to the first element, the second element with one or more second through-openings configured to receive the suture, a mechanism coupled to the first element and to the second element and configured to control relative positioning of the first and second elements between a closed configuration in which at least one of the one or more first through-openings is aligned with at least one of the one or more second through-openings and in which the first and second elements sandwich the implantable patch between the first and second elements, and an open configuration in which the implantable patch is not sandwiched between the first and second elements. According to some embodiments of the present invention, the mechanism is a hinge, which may have a hinge axis about which the first and second elements may pivot with respect to each other. According to embodiments of the present invention, the first element includes a first edge and a second edge opposite the first edge, the second element includes a third edge and a fourth edge opposite the third edge, and the hinge couples the first and third edges. In such cases, the device may further include a locking mechanism that releasably couples the second and fourth edges in the closed configuration.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a perspective view of a system for peroperative preparation of an implantable suture patch in a fully open position, according to embodiments of the present invention.

FIG. 2 illustrates a perspective view of the system of FIG. 1 in a partially closed position, according to embodiments of the present invention.

FIG. 3 illustrates a perspective view of the system of FIG. 1 in a fully closed position with an implantable patch retained therein and suture passer needles used to pass suture through the patch, according to embodiments of the present invention.

FIG. 4 illustrates a perspective view of the system of FIG. 1 with sutures passed through openings in the device, according to embodiments of the present invention.

FIG. 5 illustrates a side cross sectional view of the system of FIG. 4 taken along plane V of FIG. 4, according to embodiments of the present invention.

FIG. 6 illustrates a perspective view of the system of FIG. 1 in a fully closed position, according to embodiments of the present invention.

FIG. 7 illustrates a perspective view of another system for peroperative preparation of an implantable suture patch in an open position, according to embodiments of the present invention.

FIG. 8 illustrates a perspective view of the system of FIG. 7 in a fully closed position, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

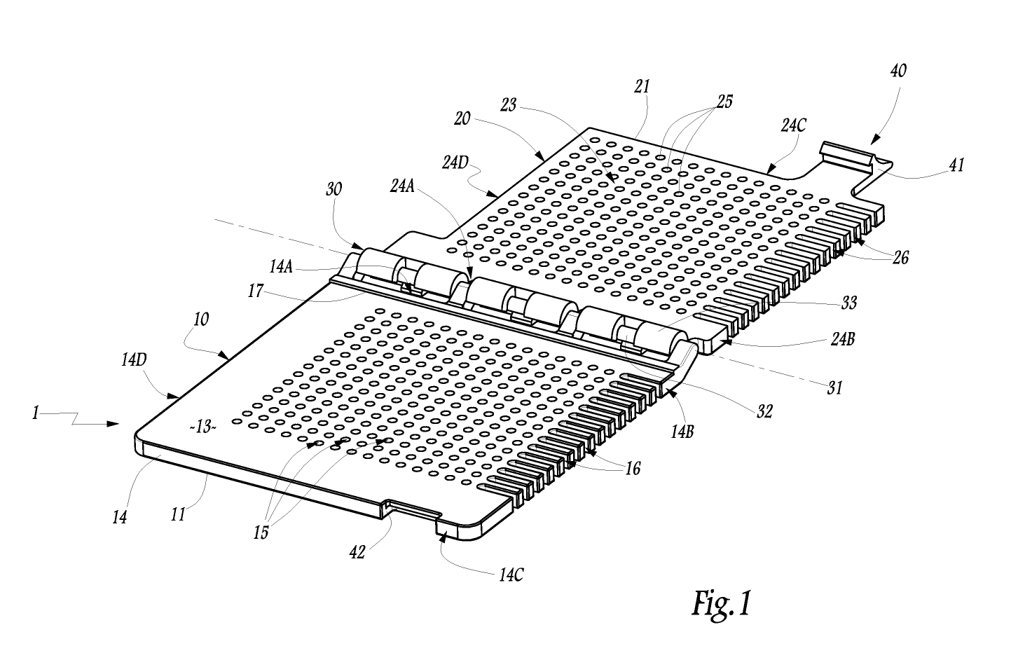

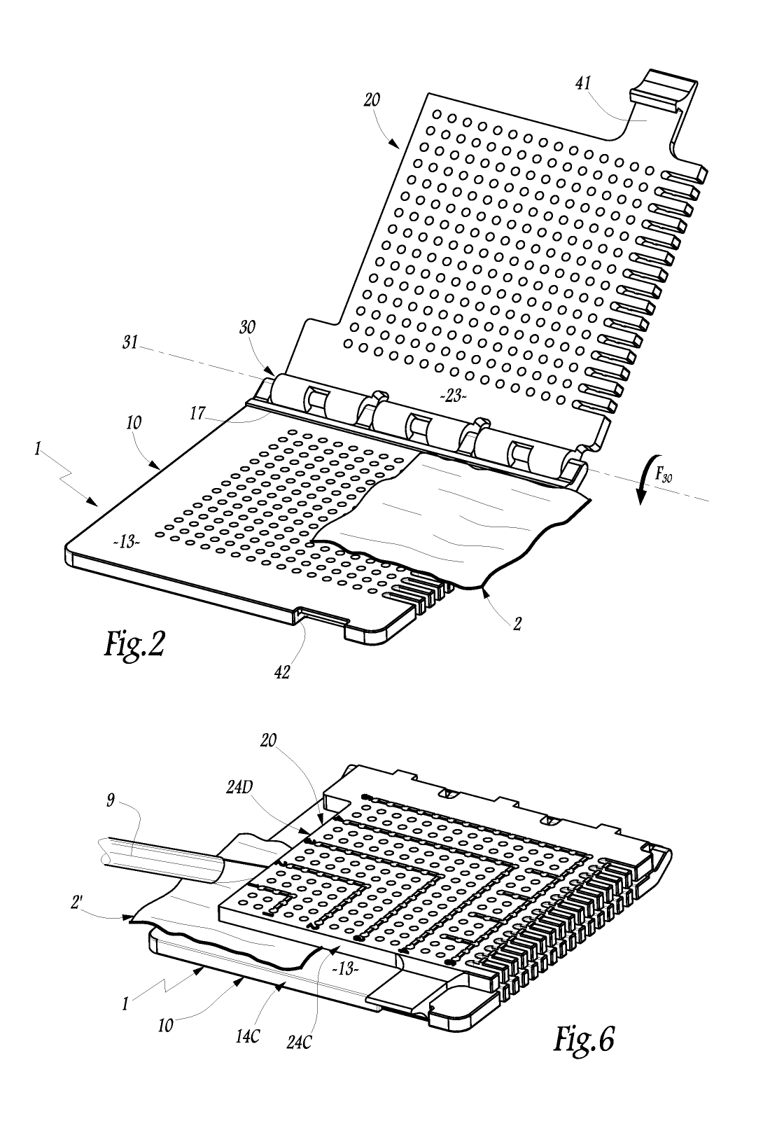

FIGS. 1 to 6 illustrate a device 1 for preparing an implantable patch 2, which is at least partially visible in FIGS. 2 to 5, according to embodiments of the present invention. As used herein, the term “patch” is used in its broadest sense to refer to transplants or partial grafts, comprising biocompatible material, whether biologic or synthetic. Such patches are used more and more often to reconstruct an original tissue of a patient, which has undergone alteration or trauma. Also, such patches are actually used, for example, in orthopedic surgery to reconstruct an articular surface of a bone, as well as in ligament surgery to repair tendons or muscular attachments, as well as the attachment of the rotator cuff, according to embodiments of the present invention. Patch 2 may be a sheet adapted to be implanted in the tissue of the body of a patient during a surgical procedure, in order to reconstruct or replace all or part of an original anatomic tissue of the patient, as well as a bony tissue, a ligament, and/or the like, according to embodiments of the present invention. The patch 2 may be formed of a biocompatible material, which may be nonresorbable if necessary, and either biologic or synthetic. In particular, the sheet of the patch 2 is made of a tissue matrix of animal origin, for example porcine origin, or of human origin, or synthetic, according to embodiments of the present invention.

As shown in FIG. 1, the device 1 includes two elements: element 10 and element 20, which may each be shaped like a board, and which are connected to each other by a hinge joint 30. Each element 10, 20 includes a body 11, 21, which may have an overall shape of a parallelepiped of which the thickness is significantly smaller than the length and width and which, in the example shown in FIG. 1, is uniform in its formation (e.g. formed as a single component). As illustrated in FIG. 5, body 11 includes an inner surface 13 and an outer surface 12, and body 21 includes an inner surface 23 and an outer surface 22, according to embodiments of the present invention. Each top and bottom surface is separated from the other by the thickness of the respective body 11, 21, in forming, along the perimeter of the body, an outer edge 14, 24. In the example shown, each border 14, 24 includes four global rectangular portions, which encircle the perimeter of the body 11, 21. Each body 11, 21, may be formed of a transparent and/or translucent material, according to embodiments of the present invention.

The mechanism 30 connects respective portions 14A and 24A of borders 14 and 24 of the elements 10 and 20. The mechanism 30 permits elements 10 and 20 to rotate with respect to each other about hinge axis 31, which extends along a direction parallel to the longitudinal direction of border portions 14A and 24A, according to embodiments of the present invention. Various other mechanisms may be used to permit movement between elements 10 and 20. As illustrated, the portion of the border 14A includes a rod 32 centered on the axis of rotation or hinge axis 31, and around which is mounted, in a pivoting manner, a clip 33 which is part of the border portion 24A, according to embodiments of the present invention. The particulars of this mechanism 30 are illustrative, in the sense that a multitude of other configurations may be used for the hinge joint 30, according to embodiments of the present invention.

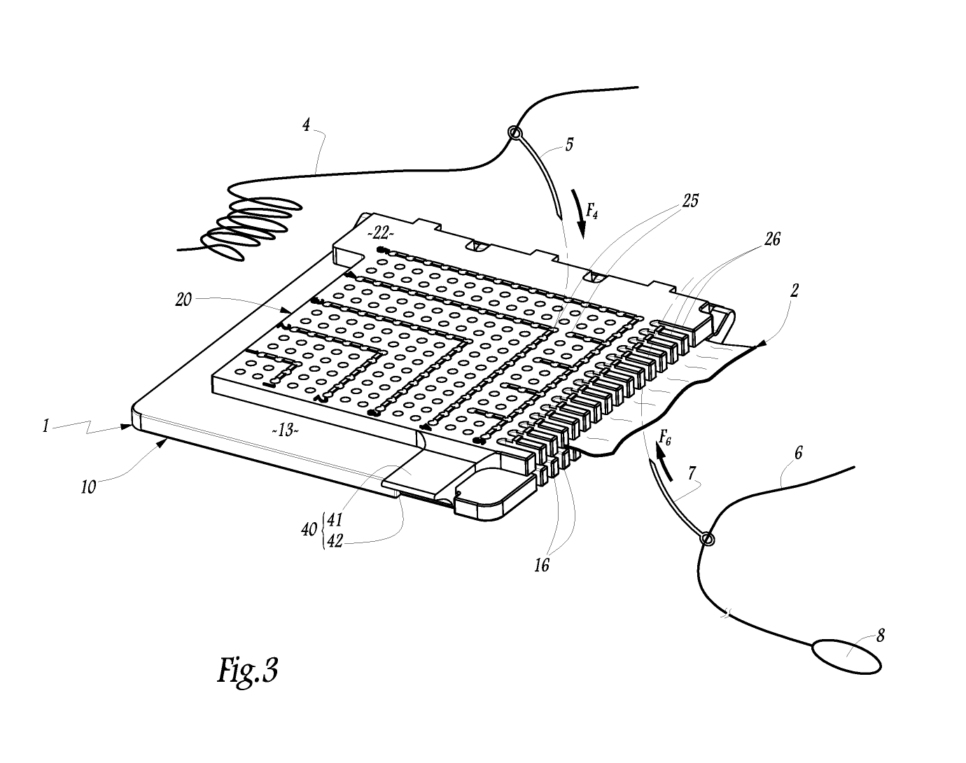

By pivoting the elements 10 and 20 about hinge joint 30, the elements 10 and 20 are moveable with respect to each other between an open position, shown in FIG. 1, and a closed position, shown in FIGS. 3 to 5, according to embodiments of the present invention. FIG. 2 illustrates an intermediate position between the closed and open positions, according to embodiments of the present invention. In the open position of FIG. 1, the elements 10 and 20 are positioned side-to-side such that their respective bodies 11, 21 extend in substantially the same plane, their respective faces 12 and 22 facing in the same direction, downwardly in the configuration shown in FIG. 1, and their respective faces 13, 23 facing in the opposite direction, upwardly in the configuration shown in FIG. 1. The open position of the device 1 shown in FIG. 1 may also be achieved by laying the surfaces 12, 22 against a horizontal work surface, according to embodiments of the present invention.

In the closed configuration of FIGS. 3 to 5, the elements 10 and 20 are positioned against each other such that their respective bodies 11, 21 extend in distinct and parallel planes, and their faces 13, 23 face in opposite directions. In this closed configuration, the face 13 of the body 11 of the element 10 is positioned against and turned toward the face 23 of the body 21 of element 20, in forming a free space 3 between the two faces 13, 23, according to embodiments of the present invention.

The free space 3 is sized to receive the patch 2, as shown in FIG. 5, according to embodiments of the present invention. In the closed configuration of the device 1, the patch 2 is interposed between the elements 10 and 20, with its two principal opposite faces respectively supported against the face 13 of body 11 of element 10 and face 23 of body 21 of element 20, according to embodiments of the present invention. In other words, the patch 2 is “sandwiched” between elements 10 and 20. In this way, in the closed configuration of the device 1, the elements 10 and 20 maintain the patch 2 in place between them, by supporting their respective faces 13 and 23 against the principal opposite faces of the patch 2, according to embodiments of the present invention.

According to one embodiment of the present invention, one or the other of faces 13 and 23 is textured or, more generally, provided with bumps and/or hollows, in order to provide reliefs against which the patch 2 is mechanically retained by friction. In this manner, the immobilization of the patch 2 against the faces 13 and 23 is reinforced, according to embodiments of the present invention.

As shown in FIGS. 1 and 5, each element 10, 20 includes a plurality of through-holes, which may be separated into a first group of cylindrical holes 15, 25, and a second group of slots 16, 26. The holes 15, 25 and slots 16, 26 all pass directly through each body 11, 21 thus connecting the opposite faces 12 and 13, 22 and 23 of the body. The holes 15, 25 differ in that their contour is closed on itself, in a cross-sectional plane transverse to the holes; in other words in a plane parallel to the faces 12 and 13, 22 and 23, the cross section of the hole is encircled, about its perimeter, by the material out of which the body 11, 21 is made, according to embodiments of the present invention.

The slots 16, 26 have an open contour in a transverse cross-sectional plane, their cross-section being only partially defined, along the perimeter of that section, by the material out of which the body 11, 21 is made, a peripheral portion of that section being freely open to the exterior of the body 11, 21. In other words, in addition to opening on the faces 12 and 13, 22 and 23 of the body 11, 21, the slots 16, 26 open onto the border 14, 24 of the body 11, 21. For example, the slots 16, 26 open as well on portions 14B, 24B of the border 14, 24 which are distinct from the portions 14A, 24A of the border which are adjacent to the hinge 30.

While the device 1 is in the closed configuration of FIGS. 3 to 5, the holes 15 of element 10 are aligned with the holes 25 of element 20, according to embodiments of the present invention. In other words, each one of the holes 15 is associated with a hole 25, forming a pair of holes aligned with each other. In this way, holes 15 and 25 are each an extension of the other, in particular a rectilinear extension of the other, along a direction perpendicular to the planes in which the elements 10 and 20 extend. In the same manner, in the closed configuration of FIG. 3, the slots 16 are aligned with the slots 26, according to embodiments of the present invention. In FIG. 3, each of the slots 16 is situated in the extension of one of the slots 26, in particular along the direction perpendicular to the planes of elements 10 and 20, according to embodiments of the present invention.

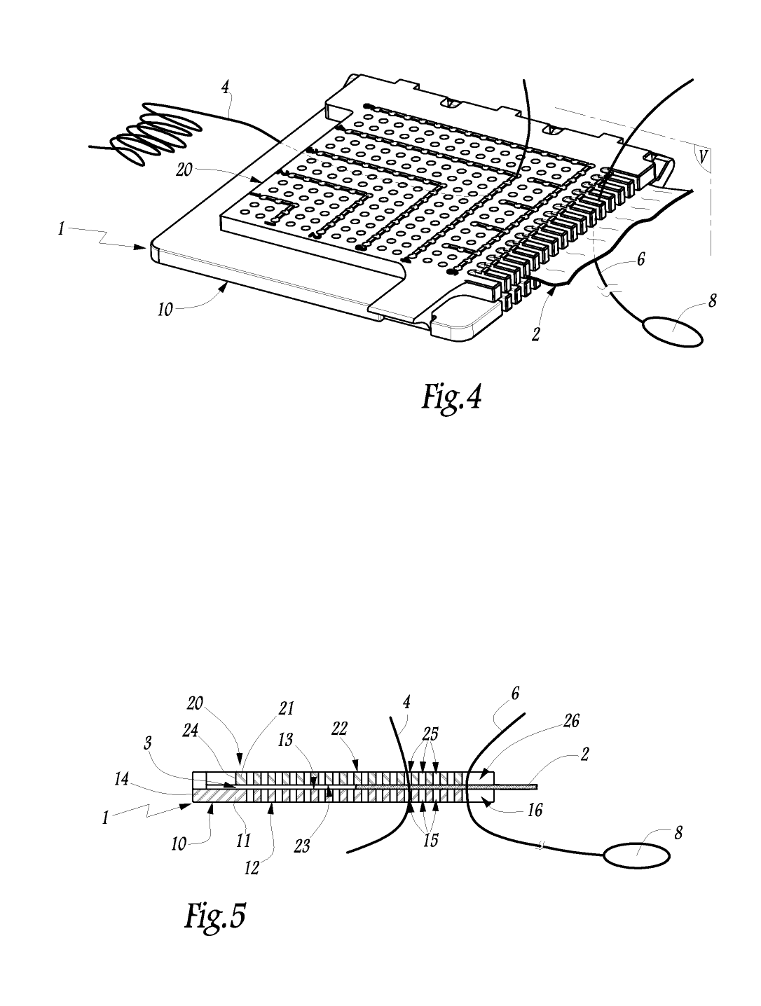

In the device 1 shown in FIGS. 3 and 4, the holes 15, 25 of each element 10, 20 are arranged or demarcated according to a measurement grid, according to embodiments of the present invention. The holes 15, 25 are marked according to a pattern in which successive lines or sets of lines are offset from each other, for example by a regular or preset distance, to facilitate the surgeon's correct sizing or stitching. The spacing between each set of lines may be quantified, for example using the numbers “1” through “5” as illustrated in FIGS. 3 and 5. Such numbers may be printed or inscribed on the body 11, 21, for example on the face 12, 22 of the body, according to embodiments of the present invention.

With respect to slots 16 and 26, they may be spaced according to a regular interval along some or all of the length of border 14B and 24B of the body 11 and 21. Also, the slots 16 and the slots 26 may be arranged in a pre-established pattern according to graduated measurements, with the distance between successive slots along the border portions 14B and 24B being pre-established, according to embodiments of the present invention.

In practice, initially, when the surgical intervention is being performed, and after the surgeon has approached the site of the implantation of the patch 2, for example with an incision or an arthroscopic passage, the surgeon places the device 1 on a work surface, in its open configuration (as shown in FIG. 1), according to embodiments of the present invention. At this stage, the device 1 is prepared according to an aseptic technique, whether the device 1 is a disposable device which is delivered to the surgeon in a sterile condition, or whether the device 1 is adapted to be reused and has been sterilized prior to surgery.

When the device 1 is in its open configuration, the surgeon takes the patch 2, when it leaves its sterile condition, and poses the patch 2 on the face 13 of the body 11 of element 10, as shown in FIG. 2. In the configuration of the device 1 shown in FIG. 2, the positioning of the patch 2 on the face 13 is facilitated by an edge 17 extending alongside the face 13, along the length of at least a part of the face 13, for example the length of the border portion 14A. Edge 17 forms an abutment for positioning the patch 2, against which the surgeon may apply the section of patch to ensure a predetermined relative positioning between the patch 2 and the face 13, but which extends in an adjoining manner the length of the edge 17, as shown in FIG. 2, according to embodiments of the present invention. Edge 17 is but one example of the implementation of an abutment for precision positioning of the patch 2, according to embodiments of the present invention.

As shown in FIG. 2, the surgeon then brings the elements 10 and 20 closer together, in pivoting the element 20 with respect to the element 10 about the hinge axis 31, as shown by the arrow F₃₀ of FIG. 2, according to embodiments of the present invention. The mechanism 30 guides the movement of the pivoting of the element 20 with respect to the element 10, until the elements 10, 20 attain the closed position of the device 1, as shown in FIGS. 3 to 5, according to embodiments of the present invention.

According to one embodiment of the present invention, a locking mechanism may be used to lock the elements 10 and 20 in the closed position. This locking mechanism 40 may take many forms. For example, the mechanism 40 includes a tongue 41 which is integral with the body 21, projecting from the border portion 24C opposite to the border portion 24A, and which is adapted to cooperate by flexible deformation relative and using complementing shapes, with a corresponding notch 42 formed in the body 11, along the border portion 14C opposite from border portion 14A, according to embodiments of the present invention. In other words, the tongue 41 is adapted to clip into the notch 42 while the device 1 is in the closed position, immobilizing the elements 10 and 20 with respect to each other, unless a specific unlocking action is performed on the tongue 41 to disengage the notch 42 and to rotate element 20 in a direction opposite to that indicated in FIG. 2, according to embodiments of the present invention.

When the device 1 is placed into the closed position, as shown in FIG. 3, the patch 2 is located interposed between elements 10 and 20, with the face 13 of the element 10 and the face 23 of the element 20 pushing against the primary opposed faces of the patch 2, according to embodiments of the present invention. The stacking of the bodies 11 and 21 one on top of the other maintains the patch 2 in place.

The surgeon then takes a first suture 4 and passes it through the patch 2, introducing it successively through one of the holes 25 and the corresponding hole 15, according to embodiments of the present invention. The diameter of the suture 4 may be smaller than the diameter of the holes 15 and 25 to permit the passage of the suture 4 therethrough. The surgeon may also use a needle 5 to pass the suture through the holes 15 and 25 and patch 2, according to embodiments of the present invention. The suture 4 may be connected to the needle 5 so as to permit passage of the suture 4 along with the needle 5 as shown by arrow F₄ of FIG. 3, according to embodiments of the present invention. FIGS. 4 and 5 illustrate this setup after the suture 4 has been placed through the patch 2 and through holes 15 and 25, according to embodiments of the present invention.

The surgeon may also use a second suture 6, independent of suture 4. As shown in FIGS. 3 to 5, the suture 6 is used in substantially the same way as suture 4, for example using a needle 7, except the suture 6 is intended for successive passage through one of the slots 16, through the patch 2, and out through the slot 26 corresponding to (e.g. in alignment with) the first slot 16. This procedure is indicated by arrow F₆ of FIG. 3, according to embodiments of the present invention. The manipulation and visual perception of the needles 5 and 7, for passing them through the elements 10 and 20, is facilitated by the fact that the bodies 11, 21 are transparent or translucent, according to embodiments of the present invention.

Although FIG. 3 illustrates sutures 4 and 6 passing through device 1 and patch 2 in certain directions, the sutures 4 and/or 6 may alternatively be passed through device 1 and patch 2 in directions opposite to those shown. In fact, face 12 may have markings similar to face 22 to permit the surgeon to align the stitches and/or cutting while looking at either face 12 or face 22, according to embodiments of the present invention.

Suture 4 is a suture which is completely free, such that having passed through patch 2, the suture 4 is totally independent of all other objects. In other words, the suture 4 resumes the character of a filamentous segment. On the other hand, the suture 6 is, at one of its ends, already connected to an object 8 before being passed through the patch 2, according to embodiments of the present invention. The object 8 may include a transverse dimension larger than the diameter of the suture 6, which explains why the suture must be passed through one of the pairs of slots 16 and 26, instead of through a pair of holes 15 and 25, according to embodiments of the present invention.

The large number of holes 15, 25 and slots 16, 26 give the surgeon a high degree of choice in placing the sutures 4 and 6 through the patch 2, according to embodiments of the present invention. The surgeon may take advantage of the markings of the square grid of the holes 15, 25 to choose with precision the positioning of the suture 4 passage through the patch 2 that takes into account how and where the patch 2 will be used in the patient, according to embodiments of the present invention. The surgeon may use measurements taken (or informal dimensions observed) at the site of implant to map out placement of the suture passages on device 1 according to the grid, according to embodiments of the present invention.

In addition, the regularly-spaced and demarcated plurality of slots 16 and plurality of slots 26 along edges 14B and 24B permit the suture to be doubled, with the help of two pairs of aligned slots 16 and 26. In this manner, the slots 16 and 26 may be used to create mattress stitches and other complex stitches in the patch 2, according to embodiments of the present invention.

After placing the sutures 4 and/or 6 and the desired suture stitches through patch 2, the surgeon may withdraw the patch 2 with the sutures 4, 6 passing through. To do this, the surgeon unlocks the mechanism 40 and pivots the element 20 with respect to the element 10 about axis 31, so as to change the device 1 from a closed configuration to an open or partially open configuration. The surgeon may then take the patch 2 and separate it from the device 1, while maintaining the sutures 4 and 6 through the patch 2. As for suture 4, its free ends may be slid out through holes 15 and 25 as patch 2 is withdrawn from device 1, thereby completely disengaging the suture 4 from elements 10 and 20, according to embodiments of the present invention. As for suture 6, the fact that the slots 16, 26 are open along outside edges 16 and 26 permits the patch 2 to be withdrawn from the device 1 with suture 6 therethrough, even while suture 6 also remains attached to object 8, according to embodiments of the present invention. This would not be possible if suture 6 had been passed through a set of closed holes 15, 25 and also an object 8, according to embodiments of the present invention. The inverse may also be performed: a suture similar to suture 4, with two free ends, may be passed through the slots 16 and 26 and may be withdrawn from device 1 along with patch 2 in a manner similar to suture 6, by sliding the suture out of the slots 16, 26, according to embodiments of the present invention.

FIG. 6 illustrates the use of device 1 for resizing a patch 2′, which may be similar to patch 2, according to embodiments of the present invention. The patch 2′ may be placed in the device 1 substantially in the same fashion as patch 2, except the patch 2′ may be positioned between elements 10 and 20 so as to extend not from portions of edges 14B and 24B like patch 2, but from portions of edges 14C and 24C, as well as from portions of edges 14D and 24D, opposite to portions of edges 14B and 24B. This positioning of the patch 2′ is helpful for the surgeon because, in this closed position, the portions of the edges 24C and 24D of element 20 do not extend all the way to edges 14C and 14D, respectively. As such, when in the closed position, the face 13 of the body 11 of element 10 does not completely cover the face 23 of body 11, leaving a portion of face 13 uncovered as shown in FIG. 6. As a result, portions of edges 24C and 24D are disposed over the face 13 of element 10, with the patch 2′ interposed between them. With the help of an adequate fit, these edge portions 24C and 24D may then be used to guide the application of a cutting tool 9, such as a scalpel or a scissors, permitting the peroperative resizing of the patch 2′, according to embodiments of the present invention.

The resizing of the patch 2′ may be easily quantified by the surgeon with the help of the quarter-square grid formed over holes 25 of element 20. In the example shown in FIG. 6, the application of the cutting tool 9 along the edge portions 24C and 24D permits the cutting, from the initial patch 2′, of a piece of rectangular patch having a length of three units and a width of two units, according to embodiments of the present invention. Once the piece of patch 2′ is cut, it may be prepared and/or sutured in the same manner as patch 2 with respect to FIGS. 1 to 5, with any necessary repositioning after opening the device 1 and before closing it again, according to embodiments of the present invention.

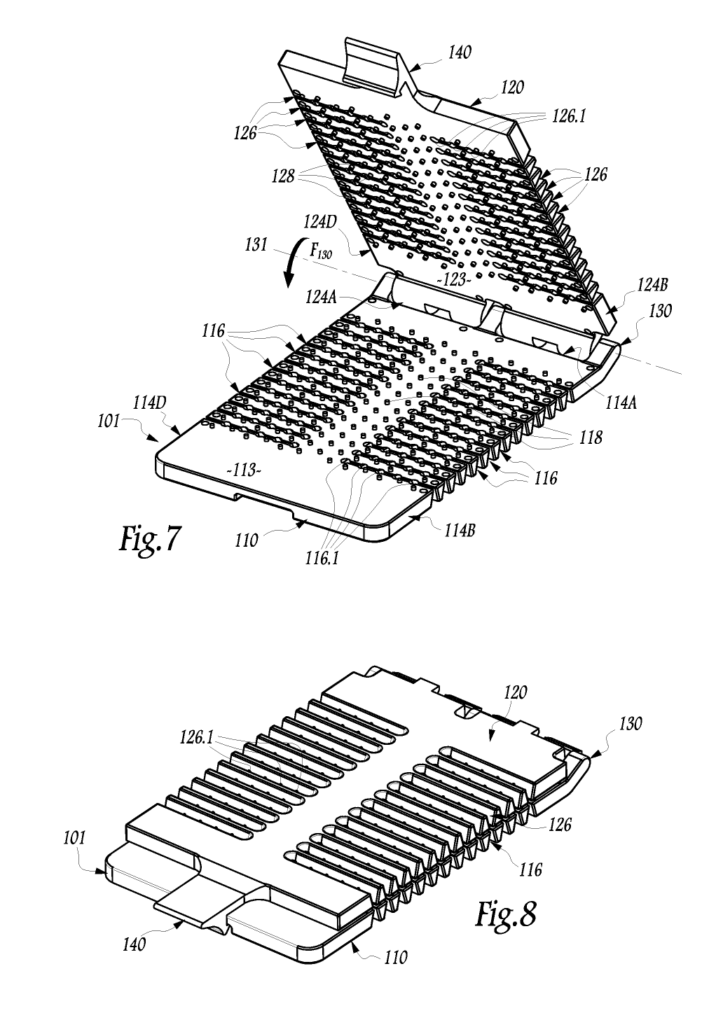

FIGS. 7 and 8 illustrate an alternative embodiment of a template device 101, according to embodiments of the present invention. Device 101 achieves similar goals with respect to device 1, namely to prepare an implantable patch similar to patches 2 and 2′, described above. For simplification, device 101 is shown in FIGS. 7 and 8 without an associated implantation patch.

Similar to device 1, device 101 includes elements 110 and 120, respectively in the form of boards, which are connected together by a hinge mechanism 130. By actuating mechanism 130, the elements 110 and 120 are moveable with respect to each other between an open configuration and a closed configuration, similar to the open and closed configurations of device 1, according to embodiments of the present invention. FIG. 7 illustrates device 101 in a partially open (e.g. intermediate) configuration, and FIG. 8 illustrates device 101 in a closed configuration, according to embodiments of the present invention. The rotation of element 110 about element 120 defined by mechanism 130 about axis 131 is shown by arrow F₁₃₀ in FIG. 7, according to embodiments of the present invention.

Device 101 includes a locking mechanism 140 which is functionally and structurally similar to the locking mechanism 40, according to embodiments of the present invention. One difference between device 1 and device 101 resides in the nature of the transverse openings in each element 110, 120, according to embodiments of the present invention. As shown in FIG. 7, each element 110, 120 lacks cylindrical holes such as holes 15, 25 of device 1, but is provided with slots 116, 126 which are functionally similar to the respective slots 16 and 26 of device 1, according to embodiments of the present invention. Instead of bordering only edges 16, 26 as in device 1, the slots 116, 126 may be discussed in two distinct groups: a first group of slots which opens onto the same edge portion 1148, 1248 of the border 114, 124 of the elements 110, 120, and a second group of slots which opens onto the same edge portion 114D, 124D of the border 114, 124 which is opposite to edges 114B, 124B, according to embodiments of the present invention.

For each of the two groups of slots 116 and 126, each of the slots may be arranged in a regularly spaced manner along the length of portions of the corresponding edges 114B, 114D, 124B, and 124D. Also, each group of slots may be provided with a graduated measurement along the length of the corresponding edge portions, such that the lengthwise distance between two successive slots is pre-established, according to embodiments of the present invention.

Another difference between devices 1 and 101 relates to the longitudinal dimension of slots 116 and 126, compared to the longitudinal dimension of slots 16 and 26, according to embodiments of the present invention. As illustrated in FIGS. 7 and 8, each slot 116 and 126 extends, from the corresponding edge portion 114B, 114D, 124B, or 124D, along a substantial portion of the body of the corresponding element 110 or 120, according to embodiments of the present invention. According to some embodiments of the present invention, each slot 116 and 126 extends across at least a third of the width of the element 110, 120; according to other embodiments of the present invention, each slot 116 and 126 extends across at least a half of the width of the element 110, 120 along the direction connecting the border portions 114B and 114D or along the direction connecting the border portions 124B and 124D.

According to one embodiment of the present invention, given the larger longitudinal extent and length of slots 116 and 126, each slot is provided with graduations 116.1, 126.1 distributed evenly along the longitudinal direction of the slot. As illustrated in FIGS. 7 and 8, these graduations 116.1 and 126.1 include a series of five hollowed divots formed in the opposing edges of the corresponding slot 116, 126, according to embodiments of the present invention. In measuring the distance between these graduations, which follows the length of the corresponding slot 116, 126 and may be pre-established, the surgeon perceives a visual indicator to permit the surgeon to place the suture through the patch 2 at the proper location, according to embodiments of the present invention.

In addition, the surfaces 113 and 123 may, in the closed position, “sandwich” the patch within the device 101. Projecting barbs 118 and 128 may be formed on or attached to elements 110, 120 in order to better grip the patch within the device 101 and to prevent undesired sliding or disengagement in the closed position, according to embodiments of the present invention. The barbs 118, 128 may be distributed throughout all or a portion of each face 113, 123, and may be located in a space between the first and second group of slots 116, 126 and/or in a space between two adjacent slots of one of the groups of slots 116, 126, according to embodiments of the present invention. In this way, a patch held in device 101 is firmly retained by friction by the faces 113 and 123, according to embodiments of the present invention.

The use of device 101 is similar to that of device 1, according to embodiments of the present invention. In particular, in the closed configuration of FIG. 8, each of the slots 116 is aligned with one of the slots 126 such that the slots 116 and 126 align with each other. Sutures similar to suture 4 and/or 6 may be passed through slots 116, 126 (and also through patch) in a manner similar to that described above, according to embodiments of the present invention. The graduations 116.1 and 126.1 help the surgeon to locate in space, with respect to the patch maintained between elements 110 and 120, the placement of the passage of the suture through the patch, according to embodiments of the present invention.

Various alternative arrangements and variations of devices 1 and 101 may be used. For example, the elements 10 and 20, as well as the elements 110 and 120, may have shapes other than rectangular boards, which are capable of coming together to maintain a patch between them for preparation. The thicknesses of elements 10 and 20, as well as elements 110 and 120, may be non-uniform in some cases. Also, one or more edges of elements 10 and 20, as well as elements 110 and 120, may be non-straight; for example, they may have a circular, semi-circular, jagged, triangular, or other shape of edge.

Also, the control for the relative positioning between the elements 10 and 20, as well as between the elements 110 and 120, may be achieved with mechanisms other than hinge mechanism 30 or 130, while still providing for an open position in which a patch may be placed onto the device 1, 101, and a closed position in which the patch is retained well enough within the device 1, 101 to permit suturing and/or resizing of the patch, according to embodiments of the present invention.

Although the surfaces 13, 23 which contact the patch are described as being planar, these surfaces may alternatively have other geometries, for example complementary three-dimensional surface geometries which maintain patch placement, according to embodiments of the present invention. And although round holes 15, 25 and slots 16, 26, 116, 126 are described, openings having other geometries may be formed in the elements 10, 20, 110, and/or 120, according to embodiments of the present invention. For example, other shapes, either closed or open to one of the edges of the device, may be used. For example, multilobed holes may be used, such as a hole in the form of a cross or a plus sign “+,” and other hole designs which permit the surgeon to pass one or several suture strands through the patch, connected by specific suture stitches, such as a Mason-Allen stitch or other stitches, according to embodiments of the present invention.

Also, the number and/or spacing and/or size of through-holes or slots in elements 10 and 20, or 110 and 120, may be modified or varied; for example, each of elements 10 and 20, or 110 and 120, may be provided with a single cylindrical hole or a single slot opening to an edge, according to embodiments of the present invention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A device for peroperative preparation of an implantable patch with suturing, the device comprising:
a first element comprising one or more first through-openings configured to receive a suture;
a second element movable with respect to the first element, the second element comprising one or more second through-openings configured to receive the suture; and
a mechanism configured to position the first and second elements with respect to each other in a closed configuration in which at least one of the one or more first through-openings is aligned with at least one of the one or more second through-openings and in which the first and second elements are close enough together to hold the implantable patch between the first and second elements.

2. The device of claim 1, wherein one or both of the first and second elements is formed of a transparent or translucent material.

3. The device of claim 1, wherein the at least one of the one or more first through-openings and the at least one of the one or more second through-openings has a closed shape.

4. The device of claim 1, wherein the at least one of the one or more first through-openings and the at least one of the one or more second through-openings have an open shape and open toward an outer perimeter of the first or second elements.

5. The device of claim 1, wherein the first element comprises a first border portion, wherein the second element comprises a second border portion configured for alignment with the first border portion in the closed configuration, wherein the at least one of the one or more first through-openings has an open shape and opens toward the first border portion, and wherein the at least one of the one or more second through-openings has an open shape and opens toward the second border portion.

6. The device of claim 4, wherein the at least one of the one or more first through-openings and the at least one of the one or more second through-openings are elongated slots.

7. The device of claim 6, wherein each of the elongated slots has a length and is graduated along the length at spaced intervals.

8. The device of claim 1, wherein the one or more first through-openings is a plurality of first through-openings arranged on the first element according to a predetermined pattern, and wherein the first element comprises markings relating at least a portion of the plurality of first through-openings to the predetermine pattern to assist a surgeon in sizing the implantable patch.

9. The device of claim 1, wherein the first element comprises a first face, wherein the second element comprises a second face, and wherein the first and second faces are substantially planar and in contact with the implantable patch in the closed configuration.

10. The device of claim 9, wherein one of the first and second faces comprises an abutment edge configured to position the implantable patch with respect to the one or more first through-openings and the one or more second through-openings when an edge of the implantable patch is aligned with the abutment edge in the closed configuration.

11. The device of claim 9, wherein at least one of the first and second faces comprises retention features configured to retain the implantable patch with friction in the closed configuration.

12. The device of claim 11, wherein both the first and second faces comprise the retention features, and wherein the retention features are projecting barbs distributed across at least a majority of the first and second faces.

13. The device of claim 9, wherein the first face has a first outer perimeter, wherein the second face has a second outer perimeter, wherein the second face is not completely covered by the first face in the closed configuration, such that the first outer perimeter is configured to guide a cutting tool along the implantable patch in the closed configuration.

14. The device of claim 1, further comprising a locking mechanism configured to lock the first and second elements in the closed configuration.

15. The device of claim 1, wherein the mechanism is a hinge.

16. A system for peroperative preparation of an implantable patch with suturing, the system comprising:
a first element comprising one or more first through-openings configured to receive a suture;
a second element movable with respect to the first element, the second element comprising one or more second through-openings configured to receive the suture;
a mechanism configured to position the first and second elements with respect to each other in a closed configuration in which at least one of the one or more first through-openings is aligned with at least one of the one or more second through-openings and in which the first and second elements are close enough together to hold the implantable patch between the first and second elements;
the suture; and
at least one needle configured for placement through the at least one of the one or more first through-openings, through the implantable patch, and through the at least one of the one or more second through-openings.

17. A device for peroperative preparation of an implantable patch with suturing, the device comprising:
a first element comprising one or more first through-openings configured to receive a suture;
a second element movable with respect to the first element, the second element comprising one or more second through-openings configured to receive the suture;
a mechanism coupled to the first element and to the second element and configured to control relative positioning of the first and second elements between a closed configuration in which at least one of the one or more first through-openings is aligned with at least one of the one or more second through-openings and in which the first and second elements sandwich the implantable patch between the first and second elements, and an open configuration in which the implantable patch is not sandwiched between the first and second elements.

18. The device of claim 17, wherein the mechanism is a hinge.

19. The device of claim 18, wherein the hinge has a hinge axis, and wherein the first and second elements pivot with respect to each other about the hinge axis.

20. The device of claim 19, wherein the first element comprises a first edge and a second edge opposite the first edge, wherein the second element comprises a third edge and a fourth edge opposite the third edge, wherein the hinge couples the first and third edges, the device further comprising a locking mechanism that releasably couples the second and fourth edges in the closed configuration.
